(19) United States
(12) Patent Application Publication
(10) Pub. No.: US 2002/0192708 A1
(43) Pub. Date: Dec. 19, 2002

(54) Detection of modified amino acids by mass spectrometry

(76) Inventors: Hanno Steen, Busdorf (DE); Bernhard Kuster, Heidelberg (DE); Matthias Mann, Odense (DK)

Correspondence Address: ROPES & GRAY, ONE INTERNATIONAL PLACE, BOSTON, MA, 02110-2624, US

(21) Appl. No.: 10/056,996

(22) Filed: Oct. 25, 2001

Related U.S. Application Data
(60) Provisional application No. 60/243,251, filed on Oct. 25, 2000.

(51) Int. Cl. G01N 33/53; B01D 59/44

(52) U.S. Cl. 435/7.1; 250/282

(57) ABSTRACT

Methods and systems of applying mass spectrometry to the analysis of peptides and amino acids, especially in the proteome setting. More particularly, the invention relates to a mass spectrometry-based method for detection of amino acid modifications, such as phosphorylation.

### CONTINUING APPLICATION DATA

This application claims priority to U.S. Provisional patent application Serial No. 60/243,251 filed on Oct. 25, 2000, the specification of which is incorporated herein by reference.

### FIELD OF THE INVENTION

This invention is in the field of proteomics, and applies mass spectrometry to the analysis of peptides and amino acids. More particularly, the invention relates to a mass spectrometry-based method for detection of amino acid modifications, such as phosphorylation.

### BACKGROUND TO THE INVENTION

As complete genomic sequences of various organisms continue to be established, and the identification of gel-separated proteins is being performed routinely, there is an increasing interest in screening also for protein modifications to obtain more information than only the identity. Each additional level of information wanted increases the degree complexity and difficulty/severity of the analysis which is normally accompanied by the additional demand for sample. However, this is often not easily achieved which is generating a demand for highly sensitive and specific methods to localize protein modifications.

One of the most common modifications is protein phosphorylation. It is estimated that ⅓ of all proteins present in a mammalian cell are phosphorylated and that kinases, enzymes responsible for that phosphorylation, constitute about 1-3% of the expressed genome. A phosphate group can modify serine, threonine, tyrosine, histidine, arginine, lysine, cysteine, glutamic acid and aspartic acid residues. However, the phosphorylation of hydroxyl groups at serine (90%), threonine (10%), or tyrosine (0.05%) residues are the most prevalent, and are involved among other processes in metabolism, cell division, cell growth, and cell differentiation. Tyrosine-phosphorylation plays an important and major role, for instance, in intracellular signaling: the binding of extracellular signaling molecules such as hormones, cytokines, or neurotransmitters to a receptor activates a phosphorylation cascade, to trigger the cell to initiate the intended response to the external stimulus.

The identification of phosphorylation sites on a protein is complicated by the facts that proteins are often only partially phosphorylated and that they are often present only at very low levels. Therefore techniques for identifying phosphorylation sites should preferably work in the low picomole to sub-picomole range.

The traditional way to localize the phosphorylation site on a given protein sample to be analyzed is by first labeling the proteins with radioactive phosphorus isotopes using hot γ-ATP followed by protease treatment of the protein and two-dimensional thin-layer chromatography (TLC) to isolate one or more spots on the autoradiography. Site directed mutagenesis or mutation experiments are performed to make the spot of interest disappear so that the site of mutation can be correlated to the site of phosphorylation. Though this approach is very sensitive, it is very tedious. A more direct method entails elution of the peptide from the TLC-plate followed by Edman sequencing (T. Hunter, ME 201, 1991). However, phospho-threonine and -serine esters are hydrolyzed under the conditions used for Edman sequencing. In the latter case, the dehydroalanine formed gives blank in the cycle so that only an indirect location of the site of phosphorylation is obtained.

Also, because endogenous ATP is present in the cells, the in vivo labeling has a low efficiency. To obtain a detectable amount of labeled protein, large amounts of radioactivity are required so that additional safety requirements have to be fulfilled to reduce the danger of handling those amounts.

Mass spectrometry is becoming increasingly the method of choice for the localization of phosphorylation sites since no radioactivity is required for the detection and since it can handle peptide mixtures, omitting the step of peptide separation. Additionally, mass spectrometry is much faster and much less cumbersome.

Several mass spectrometry based techniques have been employed for the mapping of phosphorylation sites. One of those techniques utilizes the mass shift of a phosphopeptide upon loss of the phospho-group. This loss can be observed as metastable decay/ion using a matrix-assisted laser desorption ionization-time of flight (MALDI-tof) mass spectrometer (Annan Carr AC 1996). However, the presence of metastable ions is not limited to phosphopeptides and the technique as currently practiced is therefore not very specific. Alternatively, when a MALDI-ion trap mass spectrometer is used, the signal peaks derived from phosphopeptides are accompanied by a satellite signal which is 98 Da smaller than the reference signal, and corresponds to the loss of H₃PO₄. (Qin, Chait, AC 1997).

High specificity can be achieved by treating the peptide sample with phosphatase (Yip, FEBS 1992, Liao, AB 1994, Wang JBC 1993). The mass spectra of a digest before and after treatment with a phosphatase is unaltered except for the phosphorylated peptide which is shifted downwards by 80 Da due to the removal of the phosphogroup. Using a MALDI-Tof mass spectrometer for the analysis, this method can be very sensitive due to the inherent sensitivity of this kind of mass spectrometry. However, no information about the exact phosphorylation site is obtained, which is problematic when: a) several potential phosphorylation sites are present within a peptide, b) there are isobaric peptides, and c) the identity of the protein is unknown. Depending on the phosphatase used for the analysis, additional specificity can be gained to solve at least the problem of having several potential phosphorylation sites in one peptide due to the relatively low abundance of tyrosine in proteins and therefore low likelihood to have more than one tyrosine in one peptide. This was shown by Amankwa et al. (Prot Sci 199) who used a phosphotyrosine specific phosphatase to localize phosphorylated tyrosines.

Another technique effective to isolate the phosphopeptides in protein digests exploits the affinity of phosphate for Fe-cations (Andersson AB 1986). The Fe cations can be immobilized on an appropriate resin, and used to capture and then elute phosphate-bearing peptides, either off-line (Betts, JBC 1997) or online coupled with electrospray mass spectrometry (Nuwaysir JASMS 1993). As the immobilized metal affinity columns (IMAC-columns) function also as an ion exchange material, however, they have a propensity for binding also those samples with high salt content or those containing peptides that are acidic or are high in histidine content. Inconveniently, this makes further sequencing experiments necessary in order to identify the phosphopeptide unambiguously.

A third technique for the specific detection of phosphopeptides in peptide mixtures utilizes the preferred loss of the phosphogroup upon low energy collisional activation. Whereas in the positive ion mode the neutral loss of 98 Da corresponding to H₃PO₄ is observed, in negative-ion mode a fragment ion at m/z−79 is observable corresponding to PO³⁻. Covey et al. used the former one for an LC/MS approach (Covey, 1991) to identify the phosphopeptide containing fractions in the chromatogram. This method has the disadvantage that the shift observed in the mass spectrum depends on the charge state of the precursor ion, so that only a limited set of phosphopeptide can be detected in one LC/MS experiment. On the other hand, since all data are acquired in the positive ion mode, on-line sequencing by MS/MS is possible. In contrast to this is the fragment at m/z-79 independent of the charge state of the precursor ion, i.e. giving a more comprehensive picture of the abundant phosphopeptides in the peptide mixture. Combining this approach together with m/z-dependent orifice voltage settings in a LC/MS experiment, Huddlestone et al. (JASMS, 1993), and later also Ding et al. (Ding RCM 1994) were able not only to locate the phosphopeptide-containing fractions in the chromatogram but also to measure the precursor mass of the phosphopeptides. As peptide sequencing by MS/MS in the negative ion mode is not feasible, a second MS experiment is necessary (or in cases were the effluent of the LC split after the column another off-line experiment) to localize the phosphorylation site. Although sub-picomole limits of detection for protein digests (Neubauer AC 1999; Carr AAB 1996) were shown for in-solution digests (Carr AB 1996) as well as for in-gel digests (NeubauerjAC 1999), one had to balance between either compromising the sensitivity of the detection of the characteristic 79 fragment ion in the negative ion mode by using neutral buffer conditions instead of basic conditions (Wilm, AC 1996; Carr AB 1996), or risking to lose some sample due to reconstituting the sample in acidic solution after the first experiment under basic conditions.

Given the current interest in elucidating cellular signaling pathways (Akhilesh PNAS 2000), there is a need for a rapid and highly sensitive method useful to identify gel separated, silver stained proteins and to identify tyrosine phosphorylation sites at the same time.

### SUMMARY OF THE INVENTION

There has now been developed a rapid and reliable method for identifying peptides that incorporate post-translational modifications. This method utilizes mass spectrometry to identify fragments that yield a signal predicted for an amino acid carrying a modifying group. The modifying group is selected preferably from those groups incorporated intracellularly following expression of a protein, and include phosphate groups, saccharides, lipids, and the like.

In accordance with one aspect of the present invention, there is provided a method useful to identify modified amino acids within a peptide, the method comprising the steps of:

obtaining a peptide to be analyzed,

subjecting the peptide to analysis by a mass spectrometer operating in the MS/MS mode, thereby to generate a first series of precursor ions, and a second series of product ions obtained by fragmentation of selected precursor ions, and

detecting, among the fragment ions, a product ion having the signature predicted for a modified amino acid.

The method of the present invention is applied using mass spectrometers, and conditions of analysis are sensitive enough to permit the resolution of masses at the level of at least as low as about 400 parts per million (ppm), and more desirably at a level of at least as low as about 250 ppm and lower.

In embodiments of the present invention, the method is applied to identify phosphorylation sites within a peptide analyte. In a preferred embodiment of the invention, the method is applied to identify tyrosine residues that are phosphorylated.

In other embodiments of the invention, the mass spectrometer is a MALDI-TOF. In still other embodiments of the invention, the mass spectrometer is of the ESI-MS/MS type. In a preferred embodiment of the invention, the mass spectrometer is equipped with a Q2-pulsing feature enabling the selection of a certain range of fragment ion safter the fragmentation chamber and thus improving the transmission of fragments into the second MS analyzer.

In still other embodiments of the invention, the peptide sample is first obtained by treating a crude peptide sample derived from enzymatic digests of proteins, for instance obtained by immunoprecipitation with antibody to a modified amino acid of interest, to enrich for those proteins containing the modified amino acid. In a specific embodiment, the peptide sample is obtained by enzymatically cleaving proteins after immunoprecipitation of a crude protein sample using phosphotyrosine affinity agents, such as antibodies, thereby to enrich for peptides incorporating phosphotyrosine residues.

Another aspect of the invention provides a method for identifying a treatment that modulates a modification of amino acid in a target polypeptide, comprising: i) subjecting a sample containing the target polypeptide to a treatment; ii) using any of the suitable methods, determining the level of modification of amino acid in the target polypeptide, both before and after the treatment; iii) identifying a treatment that results in a change of the level of modification of amino acid after the treatment.

In one embodiment, the treatment is effected by a compound. The compound can be a growth factor, a cytokine, a hormone, or a small chemical molecule. In another embodiment, the compound is from a chemical library.

In another embodiment, the treatment is effected by temperature change.

In another embodiment, the treatment is effected by osmotic shock.

In another embodiment, the treatment is effected by pH change.

In another embodiment, the the modification is phosphorylation.

In another embodiment, the the sample is a cell.

In another embodiment, the method further comprises enriching the target polypeptide just before analyzing using suitable methods. In a preferred embodiment, the enriching is effected by immunoprecipitation of the target polypeptide.

Another aspect of the invention provides a method for identifying a modification of a polypeptide induced by a treatment, comprising: i) subjecting a sample containing at least one polypeptide for the treatment; ii) using any suitable methods as described above, determining the level of a modification of amino acid in a selected polypeptide, both before and after the treatment; iii) determining whether the treatment results in a change of the level of the modification in the selected polypeptide;, and iv) identifying the selected polypeptide as capable of being modified by the treatment.

In one embodiment, the modification is phosphorylation.

In connection with those methods, another aspect of the invention provides a method for conducting a drug discovery business, comprising: i) by suitable methods mentioned above, determining the identity of a compound that modulates a modification of amino acid in a target polypeptide; ii) conducting therapeutic profiling of the compound identified in step i), or further analogs thereof, for efficacy and toxicity in animals; and, iii) formulating a pharmaceutical preparation including one or more compounds identified in step ii) as having an acceptable therapeutic profile. Such business method can be further extended by including an additional step of establishing a distribution system for distributing the pharmaceutical preparation for sale, and may optionally include establishing a sales group for marketing the pharmaceutical preparation.

The instant invention also provides a business method comprising: i) by suitable methods mentioned above, determining the identity of a compound that modulates a modification of amino acid in a target polypeptide; ii) licensing, to a third party, the rights for further drug development of compounds that alter the level of modification of the target polypeptide.

The instant invention also provides a business method comprising: i) by suitable methods mentioned above, determining the identity of the polypeptide and the nature of the modification induced by the treatment; ii) licensing, to a third party, the rights for further drug development of compounds that alter the level of modification of the polypeptide.

Another aspect of the invention provides a drug discovery business, comprising (i) by the methods described herein, determining the identity of a phosphorylated protein; and (ii) conducting drug screening assays to identify compounds which modulate (potentiate or inhibit) the phosphorylation of the identified protein. In certain preferred embodiments, the method includes the further steps of conducting therapeutic profiling of a compound, or further analogs thereof, identified as able to modulate phosphorylation for efficacy and toxicity in animals, and may also include formulating a pharmaceutical preparation including one or more compounds identified as having an acceptable therapeutic profile. In other embodiments, third parties can be licensed the rights for further drug development of compounds that alter the level of modification of the polypeptide.

These and other aspects of the invention are now described in greater detail with reference to the accompanying drawings, in which:

### REFERENCE TO THE DRAWINGS

FIG. 1 Differentiation of fragment ions of the same nominal mass using a quadrupole-TOF instrument. (A) Partial product ion spectrum of TNLSEQ(pY)ADVYR. The signal at m/z 216.04 corresponds to the Im(pY)-ion whereas the peak at m/z 216.10 represents the b₂ fragment ion of this peptide. (B) Partial product ion spectrum of the same but non-phosphorylated peptide. Only the b₂ fragment ion is observed proving that the peak at m/z 216.04 indeed derives from the phosphorylated residue.

FIG. 2 Analysis of a protein mixture consisting of 4 proteins (single strand DNA binding protein (E. coli), human transferrin, His-tagged RrmA (E. coli), and human MAP-kinase 2). (A) Nanoelectrospray quadrupole-TOF mass spectrum of the unseparated peptide mixture. Arrows indicate the position of the expected triply and quadruply charged phosphopeptide signals. The insert shows an expanded view of the quadruply charged phosphopeptide at m/z 556.7 (marked by an arrow). (B) Precursors ion scan for 216.10 (+/−0.15 Da). This spectrum is comparable to a similar experiment on a well tuned triple quadrupole mass spectrometer. Apart from the phosphotyrosine containing peptides (marked with arrows) signals corresponding to interfering a- and b-type fragment ions can be observed. Thus, no substantial claim regarding the presence of phosphopeptides in this mixture can be made. (C) Precursor ion scan for m/z 216.04 (+/−0.02 Da). All signals observed correspond to the triply and quadruply charged phosphopeptide derived from the MAPK in its doubly (★) or singly (●) phosphorylated state.

FIG. 3 Limit of detection of the precursor ion scan for m/z 216.04 (+/−0.02 Da). The phosphopeptide LRRA(pY)LG was sprayed at a concentration of 1 fmol/μl . For the spectrum shown, 15 scans (1 min/scan) were accumulated. The dwell time was set to 50 msec and the step size was 0.5 Da.

FIG. 4 Analytical sensitivity of the 216.04 (+/−0.02 Da) precursor ion scan. 100 fmol MAPK were loaded on a 1D SDS-gel. The silver stained band was excised and in-gel digested with trypsin. The digest was desalted and concentrated on a POROS R2-column. (A) MS1-spectrum of the fraction eluted with 1 μl 25% methanol/5% formic acid. (B) Precursor-of-216.04 (+/−0.02 Da) scan. In the spectrum shown, 36 scans were accumulated (0 sec/scan). Apart from the triply charged doubly phosphorylated peptide at m/z 768.7, the triply charged monophosphorylated and the quadruply charged doubly phosphorylated species are also observed (both marked with a bullet).

FIG. 5 Sequencing of phosphotyrosine peptide. The triply charged, doubly phosphorylated peptide at m/z 768.7, detected by precursor ion scanning for 216.04 (+/−0.02 Da, FIG. 4B), was sequenced by tandem MS. Several fragment ion series corresponding to different charge states and partial loss of phosphate under the CID-conditions applied, can be observed: [symbol] : y¹⁺-fragment ions; ▪: y-pᵢ¹⁺-fragment ions; ▴: y²⁺-fragment ions; ●: y-pᵢ²⁺-fragment ions; ★: y-pᵢ³⁺-fragment ions; ⇓: bⁿ⁺ and b-pᵢⁿ⁺-fragment ions. The sequence of 16 (underlined) out of 19 amino acids including the two phosphorylation sites could be derived from the MS/MS-spectrum.

FIG. 6 Localization of a tyrosine phosphorylation site on the EGF receptor isolated by SDS-PAGE. (A) MS 1 spectrum of the 20% methanol/5% formic acid elution of the POROS R2 column. (B) Precursor ion scan for 216.04 (+/−0.02 Da), showing a single peak at m/z 772.5. Close inspection of the MS 1 spectrum in (A) revealed a triply charged peptide at m/z 772.68 Da (arrow). (C) Product ion spectrum of the triply charged phosphopeptide at m/z 772.68. The sequence of 16 out of 19 consecutive amino acids and the site of tyrosine phosphorylation could be unambiguously identified from the spectrum (GSHQISLDNPD(pY)QQDFFPK).

### DETAILED DESCRIPTION OF THE INVENTION

The current progression from genomics to proteomics is fueled by the realization that many properties of proteins (e.g., interactions, post-translational modifications) cannot be predicted from DNA sequence. The present invention provides a method useful to identify modified amino acid sites within peptide analytes. These modified amino acids are amino acids that incorporate conjugating groups including but not limited to those conjugating groups are that incorporated naturally by the cell, typically as post-translational modifications. Such conjugating groups include saccharide moieties, such as monosaccharides, disaccharides and polysaccharides, as well as phosphate groups. Such conjugating groups further include lipids and glycosaminoglycans. Other modified amino acids containing various types of conjugating groups can also be detected by the present method, including amino acids modified by iodination, bromination, nitration and sulfation, and particularly amino acids modified by phosphorylation, including phosphotyrosine.

In general, the present method is applicable for the detection of any amino acid modification that is (i) stable enough to survive the collisional dissociation in the collision cell of the mass spectrometer to give a characteristic fragment ion, and for which (ii) the elemental composition includes an large number of mass deficient elements, such as S, O, P, Br, and I, so that the exact mass of the characteristic fragment ion is shifted downward relative to ‘normal’, C-, H-, and N-rich peptide fragments, so as to be resolved by a quadruple TOF mass spectrometer.

In conjunction with the identification of modified amino acids, the present invention further enables, and embraces, the identification of peptides containing modified amino acids, and the location of those modified amino acids within such peptides.

In a preferred embodiment of the invention, the present method is applied to identify modified amino acids that are phosphorylated amino acids, including phosphotyrosine, phosphoserine, phosphothreonine, phosphohistidine, phosphoarginine, phospholysine, phosphocysteine, phosphoglutamic acid and phosphoaspartic acid.

In a specific and preferred embodiment, the present method is applied to identify phosphotyrosine residues, peptides containing them, and the location of the phosphotyrosine within such peptides.

In accordance with the present method, modified amino acid identification is performed using mass spectrometers and conditions of analysis permitting fragments, i.e., fragment ions, having the same nominal mass but different exact masses to be resolved. With the present method, exact masses can be resolved at levels at least as low as about 400 ppm, and desirably lower than about 250 ppm. Mass values of 50 milliDaltons (mDa) can be differentiated by the present method. At such a resolution, modified amino acids carrying very small conjugating groups, such as phosphate groups, e.g. the phosphotyrosine immonium ion, can be resolved from other modified amino acids and dipeptides having otherwise very similar masses.

Such sensitivity can be achieved using a variety of different mass spectrometers. In practice, these will include particularly the hybrid instruments that operate in the tandem MS/MS mode. In tandem MS/MS instruments, the peptide sample, usually in the form of a tryptic protein digest, is typically injected as an ionized electrospray into a first mass analyzer to yield a first mass spectrum of the ions present in the mixture (‘normal’ mass spectrum). Each ion can then be channeled selectively (i.e., the precursor or parent ion), into a fragmentation chamber in which fragment ions are generated from each precursor ion. The fragment ions are then moved into a second mass analyzer, to yield a mass spectrum for the fragment ions. From the mass spectra generated in the first mass analyzer, for the precursor ions, it is possible by deduction from both mass spectral databases and protein sequence databases, to identify many known proteins. However, confidence in the result for known proteins, and in the result for unknown proteins, can be confounded by the presence of modified amino acids. As shown in the table below, the confidence levels are reduced by the similarity of masses among numerous possible precursor ions:

| Fragment ion type | Amino acid composition | m/z (Da) |
|---|---|---|
| Im(pY) | | 216.043 |
| b₂ | NT | 216.098 |
| b₂ | QS | 216.098 |
| b₃ | AGS | 216.098 |
| b₃ | GGT | 216.098 |
| a₂ | EN | 216.098 |
| a₂ | DQ | 216.098 |
| a₃ | ADG | 216.098 |
| a₃ | EGG | 216.098 |
| b₂(¹³C) | DV | 216.106 |
| a₂ | GW | 216.113 |
| a₂(¹³C) | NQ | 216.118 |
| a₃(¹³C) | AGN | 216.118 |
| a₃(¹³C) | GGQ | 216.118 |
| a₄(¹³C) | AGGG | 216.118 |
| a₂ | DK | 216.135 |
| b₂ | KS | 216.135 |
| a₃ | AAT | 216.135 |
| a₃ | GSV | 216.135 |
| a₂(¹³C) | EL | 216.143 |
| b₂(¹³C) | LT | 216.143 |
| y₂(¹³C) | PV | 216.143 |
| a₂ | RS | 216.146 |
| a₂(¹³C) | NK | 216.154 |
| a₃(¹³C) | GGK | 216.154 |

The phosphotyrosine immonium ion has an m/z of 216.04 and is therefore not discernible from the listed precursor ions without highly selective and sensitive analytical procedures.

In the present method, precursor ions having a mass difference as small as 250 ppm are discerned and resolved using hybrid mass spectrometers of the MS/MS type. Useful such mass spectrometers include quadrupole time of flight (Q-TOF) mass spectrometers, particularly of the orthogonal type, and in a preferred embodiment, versions thereof that incorporate Q2-pulsing means for gating and pulsing, thereby to concentrate, the flow of fragment ions selectively into the second mass analyzer. The Q-TOF instrument shows improved speed of acquisition, accuracy and resolution compared to triple quadrupole instruments. When compared to hybrid instruments with sector field mass analyzer, the Q-TOF instrument shows far superior sensitivity, of the type useful to discern the fragment ions of interest in the present invention. The preferred instrument is a Q-TOF instrument in which the gating means is most usefully represented by the Q pulsing arrangement described in the literature (see for instance Whitehouse, C. M.; Gulcicek, E.; Andrien, B.; Banks, F.; Mancini, R. Proc. 46th ASMS Conf. Mass Spectrom. Allied Top. Orlando, Fla., 1998; 39; and Chernushevivh, I. V.; Shevchenko, A. A.; Thomson, B. Proc. 48th ASMS Conf Mass Spectrom. Allied Top. Long Beach, Calif., 2000; 1239).

With this Q2 pulsing feature, the fragment ions are trapped/slowed down in the collision cell by applying a voltage to the exit lens of the collision cell (the IQ3 voltage) which is closer to the voltage value at the entrance lens (IQ2) than it would be for normal applications. This keeps the ions in the collision cell for a user-defined period of time (the Ion Release Width), which depends on the m/z-region of interest, the exit lens voltage is lowered to release the ions into Q-TOF interface region. This is synchronized with the pusher-puller so that after a defined delay time (Ion Release Delay) which again depends on the m/z-region of interest, the fragments ions are pulsed into the tof mass analyzer. For smaller ions, which are faster and more focused, the IRW as well as the IRD-value are lower than for larger ions. As the continuous ion beam is now axially transformed into pulsed ion packages (and not perpendicularly) the number of ions going into the tof mass analyzer can be increased by a factor of about 18 increasing the duty cycle to above 90% for smaller ions with an m/z−value below 300.

In a preferred embodiment, the mass spectrometer has the properties and functionalities of the Q-Star instrument having the Q2 pulsar feature, as sold by PE-Sciex.

The peptide samples for analysis by the present invention can be obtained, and supplied to the mass spectrometer, using techniques well established in the art and not repeated herein detail. Desirably, the sample may be enriched for modified amino acid-containing proteins using affinity chromatography and an appropriate ligand, or by immunoprecipitation using antibody to the targeted peptide.

Delivery of the sample is desirably performed by electrospray, in accordance with the instructions provided by the supplier of the electrospray and mass spectrometer device(s).

Settings available on the mass spectrometer will obviously be adapted to allow the discernment of very small differences in masses of the fragment ions.

The instant invention provides valuable means of detecting polypeptide modification, especially post-translation modification (phosphorylation, etc.), in a very specific and sensitive fashion. This is very useful for a variety of applications.

For one thing, the instant invention provides a method to identify a treatment that can modulate a modification of amino acid in a target polypeptide. By comparing the level of a modification before and after certain treatments, one can identify the specific treatment that leads to the change in level of modification. To illustrate, one can screen a library of compounds, for example, small chemical compounds from a library, for their ability to induce the phosphorylation of a target polypeptide. While in other instances, it may be desirable to screen compounds for their ability to inhibit the constitutive phosphorylation of a target polypeptide (i.e., a member of the MAPK cascade phosphorylated as the result of activation of an oncoge, such as Ras).

Similar treatments are not limited to small chemical compounds. For example, a large number of known growth factors, cytokines, hormones and any other known agents known to be able to modulate post-translational modifications are also within the scope of the invention.

In addition, treatments are not limited to chemicals. Many other environmental stimuli are also known to be able to cause post-translational modifications. For example, osmotic shock may activate the p38 subfamily of MAPK and induce the phosphorylation of a number of downtream targets. Stress, such as heat shock or cold shock, many activate the JNK/SAPK subfamily of MAPK and induce the phosphorylation of a number of downtream targets. Other treatments such as pH change may also stimulate signaling pathways characterized by post-translational modification of key signaling components.

In another respect, the instant invention also provides a means to characterize the effect of certain treatments, i.e., identifying the specific post-translational modification on specific polypeptides as a result of the treatment.

To illustrate, one may wish to identify the effect of treating cells with a growth factor. More specifically, one may desire to identify the specific signal transduction pathways involved downstream of a growth factor. By comparing post-translational modification levels of certain candidate polypeptides before and after the growth factor treatment, one can use the method of the instant invention to determine precisely what downstream signaling pathways of interest are activated or down regulated. This in turn also leads to the identification of potential drug screen targets if such signaling pathways are to be modulated.

In connection with those methods, the instant invention also provides a method for conducting a drug discovery business, comprising: i) by suitable methods mentioned above, determining the identity of a compound that modulates a modification of amino acid in a target polypeptide; ii) conducting therapeutic profiling of the compound identified in step i), or further analogs thereof, for efficacy and toxicity in animals; and, iii) formulating a pharmaceutical preparation including one or more compounds identified in step ii) as having an acceptable therapeutic profile. Such business method can be further extended by including an additional step of establishing a distribution system for distributing the pharmaceutical preparation for sale, and may optionally include establishing a sales group for marketing the pharmaceutical preparation.

The instant invention also provides a business method comprising: i) by suitable methods mentioned above, determining the identity of a compound that modulates a modification of amino acid in a target polypeptide; ii) licensing, to a third party, the rights for further drug development of compounds that alter the level of modification of the target polypeptide.

The instant invention also provides a business method comprising: i) by suitable methods mentioned above, determining the identity of the polypeptide and the nature of the modification induced by the treatment; ii) licensing, to a third party, the rights for further drug development of compounds that alter the level of modification of the polypeptide.

### EXAMPLES

The present method is exemplified in the following experiments. Chemicals were obtained from Sigma (St. Louis, Mo., USA). High purity solvents used for nanoelectrospray experiments were purchased from Labscan (Dublin, Ireland). The peptide TNLSEQ(pY)ADVYR was custom made by Sigma-Genosys (Pampisford, UK). The unphosphorylated counterpart was prepared by dephosphorylation using alkaline phosphatase (Roche Diagnostics, Mannheim, Germany) in 50 mM NH₄HCO₃ at 37° C. for 1 hr. Single-strand DNA binding protein (SSB) was purchased from Stratagene, human transferrin was from Sigma and recombinant His-tagged RrmA was prepared by established techniques. Activated MAP-kinase 2 (MAPK) was purchased from Upstate Biotechnology (Waltham, Mass., USA). For in-gel digests, concentrations of 0.1 to 1 pmol MAPK were loaded onto 4-12% NuPage gels (Novex, San Diego, Calif., USA) and visualized either by colloidal Coomassie Blue staining (Colloidal Blue Staining Kit, Novex) or silver staining. In-gel reduction, alkylation and tryptic digestion was performed as described previously³⁴.

For immunoprecipitation experiments, a total of 7×10⁷ HeLa cells were grown in Dulbecco's modified Eagle Medium (DMEM) containing 10% fetal bovine serum (FBS). Cells were grown to 80% confluence and then cultured for additional 15 hr without serum. Cells were either untreated (control) or treated (experiment) with 1 μg/ml of epidermal growth factor (EGF, Upstate Biotechnology) for 5 min. and subsequently lysed in 50 mM Tris-HCl pH 7.6, 150 mM NaCl, 1% Nonidet P-40 and 1 mM sodium orthovanadate in the presence of protease inhibitors. Cleared cell lysates were incubated overnight at 4° C. with a mixture of anti-phosphotyrosine antibodies: 30 μg of 4G-10 monoclonal antibody coupled to agarose beads (Upstate Biotechnology) and 10 μg of biotin-conjugated RC20 monoclonal antibody bound to streptavidin-agarose beads (Transduction Laboratories, Lexington, Ky., USA). Precipitated immune complexes were washed three times with lysis buffer and then eluted twice with 100 mM phenyl phosphate in 1×PBS at 37° C. Proteins from control and experiment were separated by SDS-PAGE under reducing conditions. After visualizing by silver-staining bands of interest were excised and subjected to in-gel reduction, alkylation and tryptic digestion as previously described³⁴.

Mass Spectrometry

All experiments were performed on a QSTAR Pulsar quadrupole time-of-flight tandem mass spectrometer (PE Sciex, Toronto, Canada) equipped with a nanoelectrospray ion source (MDS Protana, Odense, Denmark). Precursor ion scanning experiments were acquired with a dwell time of 50 msec at a step size of 0.5 Da and with the Q2-pulsing function turned on. Nitrogen was used as the collision gas. The Q0-voltage, which determines the collision energy, was set to a value corresponding to one tenth of the m/z value of the precursor ion.

Serial dilutions of synthetic peptides were prepared at the appropriate concentrations and without additional purification in 5% formic acid/60% methanol. Protein digests were desalted and concentrated on a double column of POROS R2 and POROS oligoR3 material (Perceptive Biosystems, Framingham, Mass., USA) packed into GELoader tips (Eppendorf, Hamburg, Germany) as described previously^{30,35}. Columns were eluted in three steps (20%/40%/60% methanol in 5% formic acid respectively) directly into nanospray needles (MDS Protana) and each fraction was subjected to MS analysis. Proteins were identified by searching peptide sequence tags³⁶, derived from fragment ion spectra of selected peptides, against the non-redundant protein database maintained and updated regularly at the European Bioinformatics Institute (EBI, Hinxton, UK) using the program PepSea (MDS Protana).

Results and Discussion

Phosphotyrosine-Specific Precursor Ion Scanning on Quadrupole-TOF Instruments

Given the practical disadvantages of negative ion MS and tandem MS, the traditional −79 precursor ion scan for the detection of phosphotyrosine containing peptides followed by sample re-buffering for sequencing, was not followed. Instead, the use of the immonium ion of phosphotyrosine ((Im(pY), m/z 216.043)) was evaluated for the detection of phosphotyrosine containing peptides by precursor ion scanning on a quadrupole-TOF MS. This ion was described by Hoffmann et al. as a characteristic fragment ion for phosphotyrosine containing peptides³⁷ and Lehmann suggested the use of this fragment as a ‘reporter ion’ for tyrosine phosphorylated peptides in precursor ion scans on triple quadrupole instruments³⁸. However, the application of triple quadrupole MS for this experiment is not very specific because many well characterized a-, b-, and y-type peptide fragment ions give rise to signals at the same nominal mass of 216 Da (Table supra, nomenclature according to Roesptorff and Fohlmann³⁹ and Biemann⁴⁰). The relatively low resolution of quadrupoles precludes the differentiation of these ions and thus false positives are often encountered in respective precursor ion mass spectra.

Close inspection of the masses shown in the Table above reveals that the mass of the phosphotyrosine immonium ion is at least 50 ppm smaller than those of the closest interfering peptide fragments. As demonstrated herein, this mass difference can be easily resolved by quadrupole-TOF mass spectrometers which provide resolution in excess of 5000 (FWHM) and mass accuracy of better than 50 ppm. As high resolution is available in both MS and MS/MS mode, precursor ion scans with very accurate fragment ion selection can be obtained by scanning the first quadrupole, fragmenting all precursor ions in the collision cell in turn and subsequent appropriate data extraction from the continuously acquired product ion TOF spectra^{41, 42}. However, until recently, the applicability of the precursor ion function was compromised by a much lower transmission of small m/z species into the orthogonal TOF when compared to ion transmission in triple quadrupole instruments. This low transmission originates from the requirement that the continuous ion beam, created by (nano)electrospray ionization, has to be converted into ion packages in order to be compatible with a TOF analyzer. Conversion is achieved by orthogonally injecting ‘slices’ of the ion beam into the TOF MS. The drawback of this configuration for precursor ion scanning is that only a small fraction of the generated ions are actually transmitted into the TOF part. The duty cycle (i.e. the fraction of ions injected into the TOF part out of all ions leaving the quadrupole section) is mass dependent and decreases with decreasing m/z so that only about 5% of smaller ions reach the detector⁴³. Since typically small m/z species are used for precursor ion scanning, the overall sensitivity of this experiment is rather low. For comparison, fragment ion transmission in precursor ion scanning experiments on triple quadrupole instruments is close to 100%.

The problem of low transmission of small m/z species has for the most part been compensated for by the introduction of the Q2-pulsing function on quadrupole-TOF instruments (QSTAR Pulsar, PE Sciex). In this configuration, fragment ions are trapped in the collision cell for a user defined period of time. Subsequently, ions are released into the quadrupole-TOF interface region. This ion release is synchronized with the orthogonal injection device such that after a defined delay time fragment ions of a user-defined m/z-range are pulsed into the TOF mass analyzer. Since the continuous ion beam is now axially transformed into pulsed ion packages, transmission for the m/z region of interest can be greatly enhanced. The gain in ion transmission is more pronounced for small m/z species than for larger ions resulting in a duty cycle of up to 90% for ions of m/z <250. In turn, the limit of detection for precursor ion experiments using quadrupole time-of-flight instruments should become comparable to triple quadrupole mass spectrometers with the added advantage that due to the high resolving power and high accuracy of the TOF analyzer, selectivity and specificity should be greatly enhanced⁴⁴.

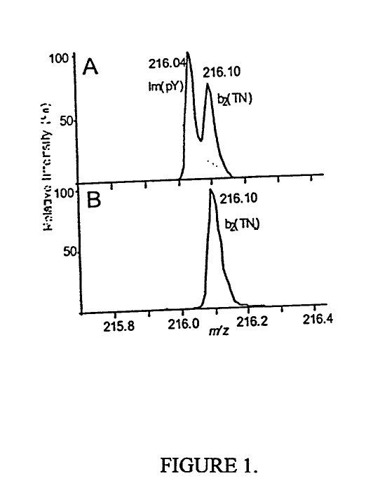

The improved precursor ion scanning capability was exploited for the analysis of tyrosine phosphorylated peptides. For this purpose, the immonium ions of phosphotyrosine (Im(pY)) at m/z 216.043 was chosen for precursor ion scanning experiments with an m/z window of +/−0.02 Da. The high accuracy fragment ion selection ensures that no peptide fragment ion (terminal or internal a-, b- and y-type ions, see table 1) of the same nominal mass would be detected. The unusually low mass increment of the Im(pY) results from the higher incident of mass deficient atoms in phosphopeptides (phosphorus and oxygen) when compared to fragment ions of non-phosphorylated peptides. In order to evaluate if the theoretical discrimination between Im(pY) and other peptide fragments of the same nominal mass would hold in practice, a synthetic peptide was designed which contained not only a phosphotyrosine residue but also TN at the N-terminus terminus. Upon low energy collisional activation, this peptide should yield an abundant b₂-ion with an exact mass of 216.098 Da which is also the closest to the exact mass of the Im(pY). The MS/MS-spectrum of this peptide (FIG. 1A) clearly shows both the fragment ion of the Im(pY) at m/z 216.04 and the b₂ ion at m/z 216.10 resolved by a 30%-valley. Performing a second experiment using the corresponding unphosphorylated peptide under the same conditions with respect to collision energy and the collision gas pressure, only the b₂ fragment ion at m/z 216.10 is detected in the MS/MS spectrum (FIG. 1B). This proves that the fragment at m/z 216.04 originates from the phosphotyrosine residue.

Specificity

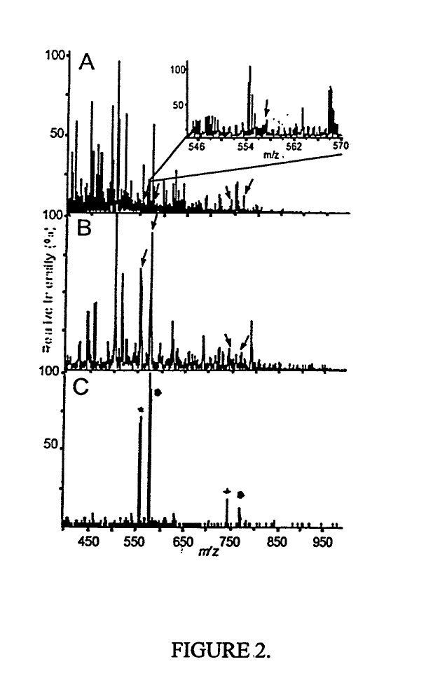

In order to address the specificity of the 216.04 precursor ion scan in analytical practice, a complex peptide mixture simulating a digest of a 200 kDa protein was prepared. The protein mixture consisting of human transferrin (79 kDa), single-strand DNA binding protein (E. coli, 19 kDa), recombinant His-tagged RrmA (E. coli, 23 kDa), and activated MAPK (68 kDa) was digested with trypsin in solution. The resulting complex peptide mixture contained a single tryptic phosphopeptide from the MAPK carrying either one or two phosphorylated residues. This mixture was used for nanoelectrospray MS at a concentration of 250 fmol/μl without any pre-fractionation. FIG. 2A shows the respective MS1-spectrum which illustrates the complexity of the peptide mixture. It is evident from the inset that the phosphopeptide signals in the region of m/z 550-580 are hardly distinguishable from the noise (the positions of the expected triply and quadruply charged phosphopeptide signals are marked with arrows).

FIG. 2B shows the result of a 216.10 (+/−0.3 Da) precursor ion scan which simulates the outcome of a similar experiment performed on a well tuned triple quadrupole MS. In this experiment, precursors of all possible a/b-type ions of m/z 216 are detected. In addition, all abundant peptides generating a/b-type ions of m/z 215 will contribute to signals in this scan since their ¹³C-isotope signals are measured at m/z 216. Although the precursor-of-216.1 experiment simplifies the spectrum considerably compared to the MS1-spectrum, most of the major signals do not correspond to phosphorylated species (the peaks corresponding to the phosphopeptides are marked with asterisks) but to interfering a- and b-type fragment ions. Thus, no substantial claim regarding the presence of phosphopeptides in this mixture can be made.

In contrast, when the accuracy of fragment ion selection for the precursor ion scan was increased to a mass window of just 0.04 Da (corresponding to the peak width at 10% height at a resolution of 5,000), only the phosphotyrosine containing peptides were detected (FIG. 2C).

The signals observed correspond to the triply and quadruply charged tryptic peptide T21 (398-416) of the MAPK containing the known TEY-phosphorylation motif of this protein with either single (asterisks) or double (bullets) phosphorylation of this motif. The inset in FIG. 2A shows that one of the largest peaks in the precursor ion scan is only of minor abundance compared to most of the other unmodified peptides in the MS1 spectrum but still gives a peak with a very high S/N ratio in the precursor ion scan. No interfering peptide was detected in FIG. 2C demonstrating the high specificity of the precursor-of-216.04 scan on a quadrupole-TOF mass spectrometer for the detection of tyrosine phosphorylated peptides.

It should be noted that both precursor ion scans could be acquired at the same time because the resulting data are derived by data processing from continuously acquired tandem MS spectra. Hence, any number of precursor ion scans could be performed at the same time.

Sensitivity

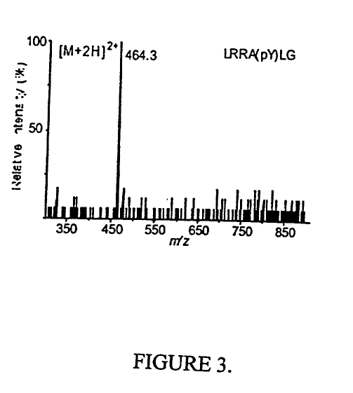

To test the sensitivity of the precursor-of-216.04 scan, a serial dilution of a synthetic peptide with the sequence LRRA(pY)LG was prepared at concentrations of 100 fmol/μl to 1 fmol/μl. As is shown in FIG. 3, even at 1 fmol/μl, a precursor ion spectrum with a S/N of greater than ten could be acquired in a reasonable period of time (15 scans, 1 min/scan, 0.5 Da step size, 50 msec dwell time). Although the peak at m/z 464.5 contains only 23 events at the detector, the very good S/N in this spectrum results the high accuracy fragment ion selection employed in the precursor ion scan (width 0.04 Da) so that the average background signal is less than one event. This limit of detection is comparable to the value reported by Wilm et al³¹. for a precursor-of(−79) scan in the negative ion mode on a triple quadrupole mass spectrometer.

However, it is rather rare that purified phosphopeptides are available for analysis. More often than not, phospho-proteins might only be available in limited quantities and contaminated with other proteins. An alternative way to looking at sensitivity of the analytical approach is to evaluate the sensitivity of the overall process. More clearly, how much of a phospho-protein needs to be present in a gel band after separation by SDS-PAGE in order to obtain information on its phosphorylation state. This question was investigated by loading defined amounts of MAPK (1 pmol to 100 fmol) onto an acrylamide gel followed by processing the protein for later phosphopeptide analysis. Protein bands were cut from the gel and in-gel digested with trypsin. Peptides were extracted from the gel plugs and concentrated in a speedvac. Subsequently, peptides were desalted on a POROS R2 microcolumn^{30,35}.peptides were eluted in three steps (1 μl of 25%, 40% and 60% methanol solutions in 5% formic acid) directly into nanoelectrospray capillaries for MS analysis.

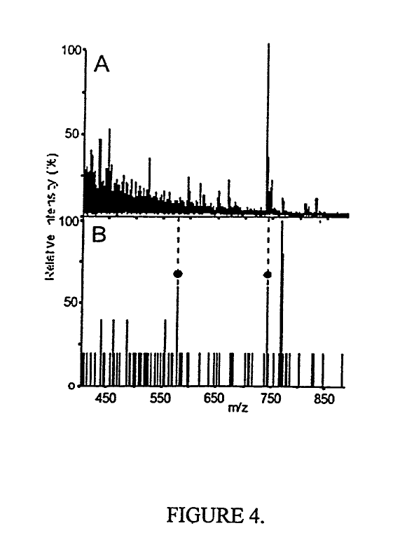

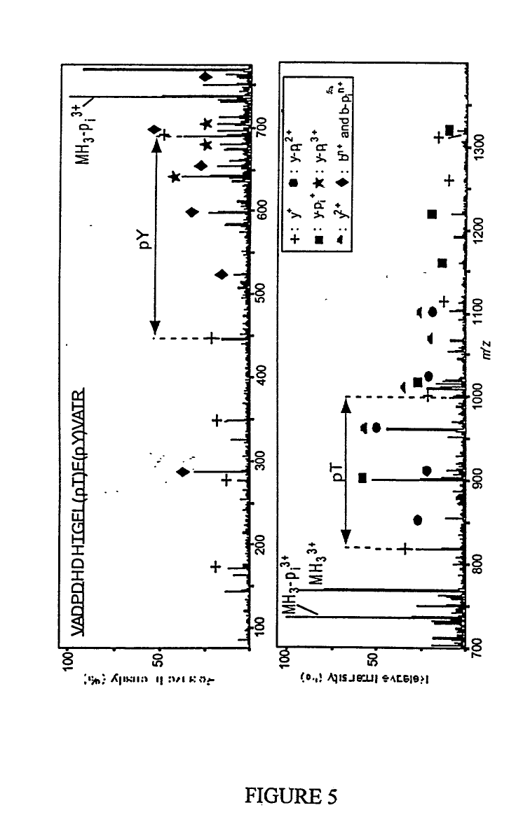

FIG. 4A shows the MS 1 spectrum of the 25% methanol fraction of the tryptic digest of 100 fmol MAPK loaded on gel. All the major peaks correspond to trypsin autolysis peaks. The precursor-of-216.04 experiment is shown in FIG. 4B. After 36 scans (50 sec/scan, 50 msec dwell time, 0.5 Da step size) a clear peak at m/z 769 was observed, corresponding to the triply charged doubly phosphorylated peptide T₃₉₈₋₄₁₆ from the MAPK. Two further signals (marked by bullets) in the precursor ion scan could be correlated with either the triply charged singly phosphorylated peptide T₃₉₈₋₄₁₆ or the quadruply charged doubly phosphorylated peptide T₃₉₈₋₄₁₆. These species were also observed in the spectrum of the in solution digest (see FIG. 2C). Close inspection of the MS1-spectrum revealed a triply charged peak at m/z 768.65. Using the same sample solution, a product ion spectrum of this peptide was acquired (FIG. 5). The spectrum is rather complex but all major peaks could be explained by standard fragmentation mechanisms revealing both phosphorylation sites and most of the sequence of this 2.3 kDa peptide. The interpretation of the product ion spectrum is somewhat complicated by the fact that most peaks are accompanied by a satellite of −98 Da due to the loss of H₃PO₄ from the phosphothreonine residue and the presence of series of singly, doubly, and triply charged fragment ions. This, experiment demonstrates that selective phosphotyrosine detection in a peptide mixture and subsequent sequencing of the tyrosine phosphorylated peptide can be performed without splitting of the same sample even in the range of 100 femtomole of protein loaded on a gel. Previous state of the art for the detection of phosphopeptides from in-gel digested proteins by the −79 precursor ion scan method was in the 250 fmol range. However, phosphorylation site determination was not possible at this level³⁰.

Application to Tyrosine Phosphorylation Mediated Receptor-Signaling Pathways

Regulatory proteins are typically of low natural abundance within a cell. Furthermore, in vivo signaling through reversible phosphorylation of proteins is often already activated at low phosphorylation stochiometries. From an analytical point of view, these two factors dictate that the analytical strategy employed must work on a low to sub picomole scale. The data shown in the previous sections demonstrate that it is now possible to obtain meaningful data at this level. To test the general applicability of the new method, an immunoprecipitation experiment was performed to isolate tyrosine phosphorylated proteins involved in the epidermal growth factor (EGF) signaling pathway. One half of a set of 3T3-cells was induced by addition of EGF and the second was not induced and served as a control experiment. Following lysis of the cells, the lysates were incubated with anti-phosphotyrosine antibody immobilized on agarose beads. Subsequent to washing the proteins bound to the immobilized antibody were eluted with phenylphosphate. After acetone precipitation, the experiment and control samples were separated by SDS-PAGE. After silver staining, bands that were clearly present in the induction experiment and absent in the control lane were cut out and processed for subsequent MS analysis as described under materials and methods. To reduce the complexity of the peptide mixture in the following mass spectrometric analysis, protein digests were loaded onto a ‘tandem-column’ consisting of a POROS R2 and a POROS oligoR3-column in a row according to the procedure described by Neubauer et al. The use of such an arrangement ensures that as few peptides as possible are lost during desalting since all small and hydrophilic peptides which are not retained by the POROS R2-column are trapped by the POROS oligoR3 material. Each column was then step eluted with 20%,40%, and 60% methanol containing 5% formic acid and each fraction was subjected to nanoelectrospray analysis.

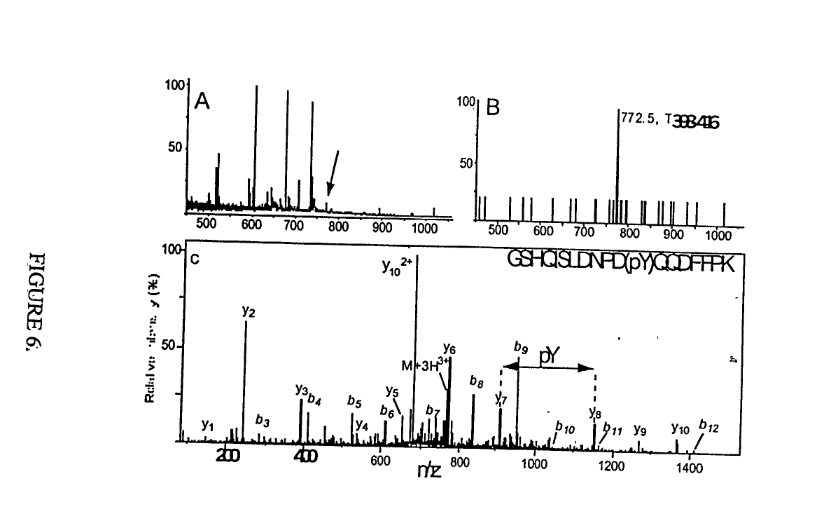

FIG. 6A shows the MS 1-spectrum of the 20% methanol fraction of the R2-column. The m/z 216 precursor ion spectrum of this fraction(FIG. 6B), exhibits one clear peak at m/z 773 indicating the presence of a tyrosine phosphorylated peptide. Close inspection of the MS1 spectrum revealed a minor triply charged peak at m/z 772.68 (indicated by an arrow in FIG. 6A). The product ion spectrum of this species is shown in FIG. 6C. Except for the three most N-terminal amino acids, the complete sequence of this 2.3 kDa peptide could be deduced from the y- and b-fragment ion series present in the MS/MS-spectrum. A peptide sequence tag was constructed from the spectrum and a database query allowing for the presence of phosphorylation identified the protein as the EGF receptor (SWISS-PROT: P0033, 130 kDa). The retrieved sequence GSHQISLDNPD(pY)QQDFFPK contains a single tyrosine residue and the data shown in FIG. 6C unambiguously confirmed the known potential phosphorylation at Tyr1172. Many other members of the EGF signaling pathway were analyzed in the same manner and the results of that study will be reported separately.

It should be noted that the m/z 216.04(+/−0.04) precursor ion scan can be acquired while other MS/MS-spectra are interpreted for ‘on-the-fly’ protein identification. Therefore, it is easily possible not only to identify a protein but also to check for tyrosine phosphorylation site in the same experiment. Furthermore, the sequence information obtained from a single tyrosine phosphorylated peptide might often be sufficient for identification of the protein and localization of the modification at the same time.

It is more generally possible, by the present method, to identify the site of a modified amino acid, by the shift in mass relative to its unmodified counterpart. This method can therefore be applied to identify various amino acid modifications, including phosphorylation, nitration, bromination, and the like. For instance, the mass shifts created by these modifications of tyrosine, relative to unmodified tyrosine and relative to each other, can readily be discerned (e.g., phosphotyrosine=216.04, nitrated tyrosine=181.061, and brominated tyrosine=237.003). Even sulfated tyrosine, which has an exact mass of 216.034 Da, too close to the mass of the phosphotyrosine immonium that discernment between the two using the present method could be difficult, doesn't interfere with the detection of phosphotyrsoine containing peptides due to the inherent low stability of the former immonium ions under low energy CID-conditions (J. Rappsilber, H. Steen, unpublished results). Also, due to the inherent lability of the phosphoesters of serine and threonine, the present method is not applicable to map phosphorylation of those residues with a similar sensitivity/limit of detection. However, the present method is also applicable to mapping phosphorylation on serine and threonine residues by scanning for the immonium ions of the respective phosphoamino acid.

Whenever the exact mass of a characteristic fragment ion differs from other fragment ions of the same nominal mass such that this difference can be resolved by a quadrupole-TOF mass spectrometer, highly specific precursor ion experiments can be performed without the risk of interference from other species. This method was applied in this study to identify phosphotyrosine containing peptides within complex peptide mixtures in a highly specific manner by using the exact mass of the immonium ion of phosphotyrosine (216.04 Da) as a reporter ion in a precursor ion scan. Unlike on triple quadrupole instruments, no unspecific signals from a-, b-, or y-type fragment ions with the same nominal mass were detected. With the recently introduced Q2-pulsing function for quadrupole time-of-flight tandem mass spectrometers, detection limits comparable to triple quadrupole mass spectrometers can be obtained. For synthetic peptides, the limit of detection is in the low fmol/μl-range whereas 100 fmol of a phosphoprotein in gel was still sufficient to identify not only the phosphotyrosine containing peptide but also to localize the site of modification. Furthermore, the method was successfully applied to the analysis of in vivo phosphorylation sites of the EGF receptor.

One of the main advantages of using the immonium ion of phosphotyrosine to detect the tyrosine phosphorylated peptides when compared to the ‘traditional’ m/z −79 precursor ion scan is that it is performed at low pH in the positive ion mode so that protein identification, phosphopeptide detection and sequencing can all be done in the same experiment, thus reducing the amount of sample and time needed for a successful analysis.

The following articles, some of which are referenced hereinabove, provide background information in the art of mass spectrometric analysis of peptides, and are incorporated herein by reference in their entirety.

### REFERENCES

(1) Hunter, T. Cell 1987, 0, 823-829.

(2) Hunter, T. In Methods in Enzymology; Hunter, T.; Sefton, B. M., Eds.; Academic Press: San Diego, Calif., 1990; Vol. 200, pp 3-37.

(3) Hubbard, M. J.; Cohen, P. Trends Biochem. Sci. 1993,18,172-177.

(4) Hunter, T. Phil. Trans. R. Soc. Lond. B 1998, 33, 83-60.

(5) Hunter, T.; Sefton, B. Proc. Nad. Acad. Sci. USA 1980, 77,1311-131.

(6) In Methods in Enzymology; Hunter, T.; Sefton, B. M., Eds.; Academic Press: San Diego, 1991; Vols. 200 and 201.

(7) van der Geer, P.; Hunter, T. Electrophoresis 1994, 1, 44-4.

(8) Yan, J.; Packer, N.; Gooley, A.; Williams, K. J. Chromatogr. A 1998,808,23-41.

(9) Aebersold, R.; Watts, J.; Morrison, H.; Bures, E. Anal. Biochem. 1991, 199, 1- 60.

(10) Meyer, H.; Hoffmann-Posorske, E.; Korte, H.; Heilmeyer, L. J. FEBSLett 1986, 204,61-66.

(11) Campbell, D.; Hardie, D.; Vulliet, P. J. Biol. Chem. 1986,261,10489-10492.

(12) Dedner, N.; Meyer, H. E.; Ashton, C.; Wildner, G. F. FEBSLett. 1988, 236, 77-82.

(13) Annan, R. S.; Carr, S. A. Anal. Chem. 1996, 68, 3413-3421.

(14) Schn6lzer, M.; Lehmann, W. Int. J Mass Spectrom. Ion Proc. 1997,169/170, 263-271.

(15) Qin, J.; Chait, B. T. Analytical Chemistry 1997, 69, 4002-4009.

(16) Yip, T.-T.; Hutchens, T. W. FEBSLett. 1992,308,149-13.

(17) Wang, K. Y.; Liao, P.-C.; Allison, J.; Gage, D. A.; Andrews, P. C.; Lubman, D. M.; Hanash, S. M.; Strahler, J. R. J. Biol. Chem. 1993, 268, 14269-14277.

(18) Liao, P.-C.; Leykam, J.; Andrews, P. C.; Gage, D. A.; Allison, J. Anal Biochem 1994,219,9-20.

(19) Zhang, X. L.; Herring, C. J.; Romano, P. R.; Szczepanowska, J.; Brzeska, H.; Hinnebusch, A. G.; Qin, J. Analytical Chemistry 1998, 70, 200-209.

(20) Cortez, D.; Wang, Y.; Qin, J.; Elledge, S. J. Science 1999, 286, 1162-1166.

(21) Andersson, L.; Porath, J. Anal. Biochem. 1986, 14, 20-24.

(22) Posewitz, M. C.; Tempst, P. Anal Chem 1999, 71, 2883-92.

(23) Betts, J. C.; Blackstock, W. P.; Ward, M. A.; Anderton, B. H. Journal of Biological Chemistry 1997, 272, 12922-12927.

(24) Nuwaysir, L. M.; Stults, J. T. J. Am. Soc. Mass Spectrom. 1993, 4, 662-669.

(25) Chicz, R.; Regnier, F. In Methods in Enzymology; Deutscher, M. P., Ed.; Academic Press: San Diego, Calif., 1990; Vol. 182, pp 392-421.

(26) Covey, T.; Shushan, B.; Bonner, R.; Schr6der, W.; Hucho, F. In Methods in protein sequence analysis; J6mvall, H., H66g, J.-O., Gustavsson, A.-M., Eds.; Birkhauser Verlag: Basel, 1991, pp 249-26.

(27) Huddleston, M. J.; Annan, R. S.; Bean, M. F.; Carr, S. A. JAm Soc Mass_Spectrom 1993, 4, 710-717.

(28) Ding, J.; Burkhart, W.; Kassel, D. B. Rapid Commun Mass Spectrom 1994, 8, 94-

(29) Carr, S. A.; Huddleston, M. J.; Annan, R. S. Anal. Biochem. 1996,239,180-192.

(30) Neubauer, G.; Mann, M. Analytical Chemistry 1999, 71, 23-242.

(31) Wilm, M.; Neubauer, G.; Mann, M. Anal Chem 1996,68,27-33.

(32) Weijland, A.; Williams, J. C.; Neubauer, G.; Courtneidge, S. A.; Wierenga, R. K.; Superti-Furga, G. Proc. Nad. Acad. Sci. 1997, 94, 390-39.

(33) Verma, R.; Annan, R. S.; Huddleston, M. J.; Carr, S. A.; Reynard; G.; Deshaies, R. J. Science 1997,4-460.

(34) Shevchenko, A.; Wilm, M.; Vorm, O.; Mann, M. Anal. Chem. 1996,68,80-88.

(35) Gobom, J.; Nordhoff, E.; Mirgorodskaya, E.; Ekman, R.; Roepstorff, P. J. Mass Spectrom. 1999,34,10-16.

(36) Mann, M.; Wilm, M. Anal. Chem. 1994,66,4390-4399.

(37) Hoffmann, R.; Wachs, W. O.; Berger, R. G.; Kalbitzer, H.-R.; Waidelich, D.; Bayer, E.; Wagner-Redeker, W.; Zeppezauer, M. Int. J. Peptide Protein Res. 199,4,26-34.

(38) Lehmann, W. D., Proceedings of the 32nd annual meeting of the German Mass Spectrometry Society, Oldenburg, 1999; 112.

(39) Roepstorff, P.; Fohlmann, J. Biomed. Mass Spectrom. 1984, 11, 601.

(40) Biemann, K. Biomed. Environs Mass Spectrom. 1988, 16, 99-111.

(41) Bateman, R.; Carruthers, R.; Hoyes, J.; Gilbert, A.; Langridge, J.; Malone, K.; Bordoli, R. Proc. 46th ASMS Conf Mass Spectrom. Allied Top. Orlando, Fla., 1998; 42.

(42) Borchers, C.; Parker, C.; Deterding, L.; Tomer, K. J. Chromatogr. A 1999, 84, 119-130.

(43) Chernushevich, I. V.; Ens, W.; Standing, K. G. Analytical Chemistry 1999, 71, 42A-461A.

(44) Whitehouse, C. M.; Gulcicek, E.; Andrien, B.; Banks, F.; Mancini, R. Proc. 46th ASMS Conf. Mass Spectrom. Allied Top. Orlando, Fla., 1998; 39.

We claim:

1. A method useful to identify modified amino acids within a peptide, comprising:
(i) obtaining a peptide to be analyzed,
(ii) subjecting the peptide to analysis by a mass spectrometer operating in the MS/MS mode, thereby generating a first series of precursor ions, and a second series of fragment ions obtained by fragmentation of selected precursor ions, and,
(iii) detecting, among the fragment ions, a fragment ion having the signature predicted for a modified amino acid, wherein the difference between the m/z of the identified fragment, and the m/z of other fragment ions is less than 250 ppm.

2. The method according to claim 1, wherein the mass spectrometer is a quadrupole time of flight mass spectrometer having a Q2 pulsing function.

3. The method according to claim 1, wherein the fragment ion is derived from a phosphorylated amino acid.

4. The method according to claim 1, wherein the fragment ion is the ammonium ion of phosphotyrosine.

5. A method useful to identify modified amino acids, comprising the steps of:
(i) obtaining a peptide digest to be analyzed;
(ii) subjecting the peptide digest, in the form of precursor ions, to a first mass analyzer of the quadrupole time of flight type to generate a first mass spectra;
(iii) subjecting a selected precursor ion for fragmentation to form a fragment ion thereof, and pulsing the fragment ion selectively into a time of flight mass analyzer, wherein the time of flight mass analyzer receives the fragment ion in an amount sufficient to resolve its mass to within not more than about 100 mDa; and
(iv) correlating the mass of the fragment ion with the mass of a reporter ion characteristic of a modified amino acid, thereby identifying the modified amino acid.

6. The method according to claim 5, wherein the reporter ion is the ammonium ion of phosphotyrosine.

7. The method according to claim 5, wherein the fragment ion is selectively pulsed to permit resolution of its mass to within not more than about 50 mDa.

8. A method for detecting phosphotyrosine in a peptide sample, comprising the steps of:
(i) subjecting the peptide sample to analysis by a Q-TOF mass spectrometer equipped with a Q2 pulsar modality; and
(ii) identifying in the mass spectra generated from the analysis, a fragment ion having an m/z of 216.04 Da.

9. The method according to claim 8, wherein the peptide sample is enriched for phosphotyrosine-containing peptides before being subjected to said analysis.

10. A method for identifying a treatment that modulates a modification of amino acid in a target polypeptide, comprising:
(i) subjecting a sample containing the target polypeptide to a treatment;
(ii) using any one of the methods in claims 1, 5, and 8, determining the level of modification of amino acid in the target polypeptide, both before and after the treatment;
(iii) identifying a treatment that results in a change of the level of modification of amino acid after the treatment.

11. The method of claim 10, wherein the treatment is effected by a compound.

12. The method of claim 11, wherein the compound is a growth factor, a cytokine, a hormone, or a small chemical molecule.

13. The method of claim 11, wherein the compound is from a chemical library.

14. The method of claim 10, wherein the treatment is effected by temperature change.

15. The method of claim 10, wherein the treatment is effected by osmotic shock.

16. The method of claim 10, wherein the treatment is effected by pH change.

17. The method of claim 10, wherein the modification is phosphorylation.

18. The method of claim 10, wherein the sample is a cell.

19. The method of claim 10, further comprising at least partially enriching the target polypeptide just before step (ii).

20. The method of claim 19, wherein the enriching is effected by immunoprecipitation of the target polypeptide.

21. A method for identifying a modification of a polypeptide induced by a treatment, comprising:
(i) subjecting a sample containing at least one polypeptide for the treatment;
(ii) using any one of the methods in claims 1, 5, and 8, determining the level of a modification of amino acid in a selected polypeptide, both before and after the treatment;
(iii) determining whether the treatment results in a change of the level of the modification in the selected polypeptide;
(iv) identifying the selected polypeptide as capable of being modified by the treatment.

22. The method of claim 21, wherein the modification is phosphorylation.

23. A method of conducting a drug discovery business, comprising:
(i) by the method of claim 11, determining the identity of a compound that modulates a modification of amino acid in a target polypeptide;
(ii) conducting therapeutic profiling of the compound identified in step (i), or further analogs thereof, for efficacy and toxicity in animals; and,
(iii) formulating a pharmaceutical preparation including one or more compounds identified in step (ii) as having an acceptable therapeutic profile.

24. The method of claim 23, including an additional step of establishing a distribution system for distributing the pharmaceutical preparation for sale, and may optionally include establishing a sales group for marketing the pharmaceutical preparation.

25. A method of conducting a business, comprising:
(i) by the method of claim 11, determining the identity of a compound that modulates a modification of amino acid in a target polypeptide;
(ii) licensing, to a third party, the rights for further drug development of compounds that alter the level of modification of the target polypeptide.

26. A method of conducting a drug discovery business, comprising:
(i) by the method of claim 21, determining the identity of the polypeptide and the nature of the modification induced by the treatment;
(ii) licensing, to a third party, the rights for further drug development of compounds that alter the level of modification of the polypeptide.

27. A method of conducting a drug discovery business, comprising:
(i) by the method of claim 1, determine the identity of a phosphorylated protein;
(ii) conduct drug screening assays to identify compounds which modulate the phosphorylation of the identified protein;
(iii) conduct therapeutic profiling of the compound identified in step (ii), or further analogs thereof, for efficacy and toxicity in animals; and
(iv) formulate a pharmaceutical preparation including one or more compounds identified in step (iii) as having an acceptable therapeutic profile.
